(19) United States
(12) Patent Application Publication
(10) Pub. No.: US 2010/0228299 A1
(43) Pub. Date: Sep. 9, 2010

(54) IMPLANT FOR FUSION OF BONES AND/OR BONE PARTS

(75) Inventors: Josef ZRINSKI, Wurmlingen (DE); Volker HAUSAM, Muehlhausen-Ehingen (DE); Stephan ECKHOF, Weilheim (DE)

Correspondence Address: COLLARD & ROE, P.C., 1077 NORTHERN BOULEVARD, ROSLYN, NY, 11576, US

(73) Assignee: ZRINSKI AG, Wurmlingen (DE)

(21) Appl. No.: 12/711,083

(22) Filed: Feb. 23, 2010

(30) Foreign Application Priority Data
Feb. 23, 2009 (DE) 202009001900.4

(51) Int. Cl. A61B 17/80

(52) U.S. Cl. 606/286

(57) ABSTRACT

In an implant for fusion of bones or bone parts, particularly of the carpal region of a wrist, as well as for application of compression between the bones or bone parts, a base body with bone screws is provided, which are guided in recesses that possess a geometry by means of which the compression between the bone, in each instance, can be adjusted, whereby the base body is configured with rotation symmetry. The geometry of its recesses has the shape of an oblong hole curved in the circumference direction of the base body, in each instance, whose curvature, with reference to the central axis of the base body, runs at an incline to this axis, in such a manner that the bone screws screwed into the bones and guided in the recesses in the manner of a motion link can be moved in the direction of the inclined oblong holes, radially toward the central axis of the base body, by means of a rotational movement of the base body.

### CROSS REFERENCE TO RELATED APPLICATIONS

This application is a U.S. Utility Application and hereby claims priority from German Application DE 20 2009 001 900.4 filed on Feb. 23, 2009, the disclosure of which is hereby incorporated by reference in its entirety.

### BACKGROUND

One embodiment of the invention relates to an implant for fusion of bones and/or bone parts, particularly of the carpal region of a wrist, as well as for application of compression between the bones and/or bone parts, having a base body with bone screws, which are guided in recesses that have a geometry by means of which the compression between the bones and/or bone parts can be adjusted. In this connection, the size of the implant is selected in such a manner that it extends at least over part of the bones and/or bone parts.

Implants of the above type serve for osteosynthesis, particularly for targeted arthrodesis of parts of the wrist. As an indication for this, among other things, acute fractures in the carpal region, but particularly degenerative arthritis as the result of old pseudarthroses of the scaphoid bone or old ruptures of the ligament connection between the scaphoid bone and the lunate bone can be present. If left untreated, these generally lead to permanent damage of the wrist, accompanied by pain symptoms.

For the transfer of muscle forces and for movement and gripping of objects, a stable and mobile wrist is absolutely necessary. Restoration of pain-free weight bearing of the hand can be achieved, with a certain restriction of movement and a reduction in the gripping force, by means of partial stiffening.

In this connection, five bones in the carpal region are of particular significance. These are the scaphoid bone, the lunate bone, the triquetral bone, the capitate bone, and the hamate bone.

In this regard, surgical intervention provides for resection of the scaphoid bone and fusion of the other four bones mentioned. For this purpose, according to the state of the art, so-called Kirschner wires and osteosynthesis plates have essentially been used up to now to fix the bones in place and to bring about osteosynthesis.

Osteosynthesis is significantly promoted by the presence of compression of the bone surfaces that touch one another. Therefore, application of a compression force is a common goal of such surgical methods. If application of a sufficient compression force is not successful, the result can be so-called pseudarthrosis. In this connection, failure of the desired bone formation to occur leads to a very painful condition.

Compression between bone segments can also take place, for example, by means of the introduction of autologous bone substance. The counterforce to interossary compression is applied by means of the surrounding ligamentous structures. This procedure is connected with an additional invasive intervention.

The compression produced in this manner must be stabilized in every case, and different methods are known from the state of the art for this purpose. One possibility consists in introducing the Kirschner wires, which have already been mentioned, out of square relative to one another, through the bones to be fused. Another possibility consists in bringing about the desired stability by means of specially shaped plates.

One of these implants is a disk-shaped element that has corresponding recesses on its surface, which are suitable for accommodating fastening means, particularly bone screws. These recesses are configured in such a manner that the fastening means can be introduced into them at a variable angle, thereby making it possible to reach the carpal bones accordingly. In this connection, screwing in the bone screws leads to a relative movement of the bone elements that are grasped, specifically toward the implant. Because of the fact that the implant is preferably configured to be conical toward the bone, an attempt is made to thereby fix the bone elements in place in centered manner.

Such implants are available in different sizes. In order to now be able to insert the implant level relative to the bone surface, the contour of the implant is prepared into the carpal bones (the corresponding bone parts are milled out).

The compression that is required to achieve osteosynthesis and also to reduce the patient's pain accordingly was not always achieved with the previous operation techniques.

In this regard, a method and a system are known from the state of the art, for example from U.S. Pat. No. 7,201,752 B2 (Jul. 22, 2003; Acumed LLC), which provide a plate for fusion of two or more bones, which plate is configured in the manner of a disk and spherical toward the bone side, wherein the teachings of this patent are hereby incorporated herein by reference.

U.S. Pat. No. 6,179,839 B1 (Sep. 20, 1999; Weiss et al.) also shows a fusion plate that is suitable for bringing multiple bone parts together, wherein the teachings of this patent is hereby incorporated herein by reference.

Furthermore, an implant for fusion of bones and/or bone parts is known from DE 20 2008 005 076 U1 (Apr. 11, 2008, Zrinski A G), which consists essentially of multiple sliding block elements, which are mounted to move within the implant, wherein the disclosure of this patent application publication is hereby incorporated herein by reference in its entirety.

Finally, there is also a tensioning device for a pressure plate for performing pressure osteosynthesis is known from EP 0 016 338 A1 (Feb. 15, 1980, Prof. Dr. Brinckmann), the disclosure of which is hereby incorporated herein by reference in its entirety.

The above cited patents have certain disadvantages. For example, the techniques that are known from the state of the art in connection with implants for wrists, in particular, all have the common disadvantage that the compression of the bones that can be achieved with them is not sufficient to achieve a satisfactory result.

Furthermore, implants are used that require conical milled-out regions in the area of the bones to be fused, but this weakens the cross-section of the corresponding bones. As a result, an additional risk of fracture can exist, particularly when the corresponding bone screws are screwed in.

Furthermore, the corresponding geometries that are known from the state of the art for achieving corresponding bone fusion by means of a bone plate are relatively complex, since they are complicated in terms of production technology and are provided with correspondingly many components.

Finally, while use of the pressure plate shown provides a solution for fusing two bone fragments, this technique cannot be easily transferred to implants used in connection with wrists, since the use of multiple separate tensioning blocks is not practicable, for reasons of space alone.

### SUMMARY

One embodiment of the invention is configured to make available an implant for fusion of bones or bone parts, particularly of the carpal region of a wrist, with the effect that in comparison with the state of the art, adjustable and, at the same time, more easily obtained compression between the bones and/or bone parts is possible.

This task is accomplished, according to at least one embodiment of the invention, in that the base body of the implant is configured with rotation symmetry. With this design, the geometry of its recesses has the shape of an oblong hole curved in the circumference of the base body, in each instance, the curvature of which, with reference to the center of the base body, runs at an incline relative to this center. In this case, the bone screws screwed into the bones are guided in the recesses in the manner of a motion link and can be moved radially toward the center of the base body, by means of a rotational movement of the base body in the direction of the inclined oblong holes.

One of the significant advantages is that the implant according to at least one embodiment of the invention has a simple design structure, and nevertheless tension forces for improved compression of bone fragments to be connected are obtained with it, in targeted manner. In this case, this is particularly brought about by the oblong holes that run in curved manner relative to the center, in which holes the bone screws are essentially compulsorily guided in the manner of a motion link.

With the contact of the implant according to at least one embodiment of the invention on the bone parts to be fused, the bone parts are seized and fixed in place by means of screwing the bone screws in at a corresponding angle direction. By means of a subsequent rotary movement exerted on the implant in the circumference direction, the screws that have been screwed into the bone parts are guided in the recesses of the implant in the manner of a motion link, so that as a result of this rotary movement, the bone parts are necessarily guided to move toward the central axis of the implant, in centered manner. As a result, the possible gaps that exist between the individual bone parts can also be reduced or cancelled out, specifically up to a point at which sufficient compression has been achieved.

To fix this compressed position in place, it can preferably be provided that in the regions of the recesses that are configured as oblong holes, in each instance, which have not become free as the result of the rotation, additional bone screws are inserted, thereby making it possible for each bone part to be fixed in place with at least two screws.

Because of the introduction of at least one additional bone screw into the residual space of the recesses, in each instance, it can therefore be prevented that unintentional unscrewing and thus decompression of the bone parts take place, and therefore fusion of the corresponding bone parts can be accelerated in very simple manner.

In another advantageous embodiment of the implant, it can be provided that the recesses of the base body are delimited, in each instance, by an edge region that possesses a surface configured to be spherical, which corresponds to part of a mantle of a sphere, thereby promoting sliding of the bone screws.

Furthermore, it is particularly practical if, in contrast to the state of the art, the base body is configured to be level, i.e. plate-like on its underside and top side, thereby eliminating any prior milling of the bone parts to be connected.

It is also practical to provide a key element for turning the implant in the clockwise or counterclockwise direction in the center of the base body. In this connection, this can be a centrally disposed recess that can accommodate a shape-fit tool and with which the implant can be put into a corresponding rotational movement. Alternatively to this, it can be provided that recesses are present on the circumference of the implant, which correspond to a tool, so that the corresponding rotation is carried out using this tool.

The configuration of the oblong-hole recesses is provided, in other words, in such a manner that their curvature, measured from the center axis toward the center line of each recess, decreases continuously in the circumference direction of the implant. In this way, the result is achieved that the bone screws are moved toward the central axis by means of a rotation of the corresponding implant, and thereby, in turn, corresponding compression of the bone parts is achieved. This again means that the curvatures of the recesses, in each instance, do not run parallel to the outside contour of the implant, which has rotation symmetry. One definition for “rotation symmetry” is that generally speaking, an object with rotational symmetry is an object that looks the same or substantially the same after a certain amount of rotation.

Alternatively to this, the curvature of the oblong-hole-shaped recesses, in each instance, can also be described in such a manner that it has a radius whose point of origin is disposed offset relative to the center axis. If the four points of origin that exist in this example are connected with one another, a square is formed.

It is also possible that the progression of the curvature of the oblong-hole-shaped recesses pivots in the region close to the central axis, on a track that is concentric to this axis or on a track that moves slightly away from the central axis. In this way, unintentional rotational loosening of the compression that has been applied is blocked. However, as a result, the bone parts to be compressed are also first guided toward one another and then moved slightly away from one another again. This means that the radius of curvature of each recess changes in the region close to the central axis.

Furthermore, it is possible that the compression takes place not only tangentially, but rather also perpendicularly. In this way, the result can be achieved that the motion link guidance of each bone screw within the recess is configured in such a manner that the bone screw is raised slightly by means of rotating the implant. In this way, the result can also be achieved that corresponding repositioning in a three-dimensional plane can be achieved.

### BRIEF DESCRIPTION OF THE DRAWINGS

Other objects and features of the present invention will become apparent from the following detailed description considered in connection with the accompanying drawings. It should be understood, however, that the drawings are designed for the purpose of illustration only and not as a definition of the limits of the invention.

In the drawings, wherein similar reference characters denote similar elements throughout the several views:

FIG. 1 a perspective view of the implant according to one embodiment of the invention, with bone screws inserted;

FIG. 2 a perspective view of the base body of the implant according to FIG. 1, on a smaller scale and without bone screws;

FIG. 3 a top view of the base body according to FIG. 2;

FIG. 4 a side view of the base body shown in FIG. 2;

FIG. 5 a cross-section through the base body according to FIG. 2, but on a larger scale;

FIG. 6 a top view for explaining the method of functioning of an implant fixed in place on four bone segments, shown schematically, in the fixation position;

FIG. 7 the top view according to FIG. 6, but here the base body is shown turned clockwise by 45 degrees as compared with FIG. 6, relative to the bone segments;

FIG. 8 the top view according to FIG. 7, but with additional bone screws to avoid decompression, i.e. as security to prevent rotation of the implant, which is already in the compressed position according to FIG. 7.

### DETAILED DESCRIPTION

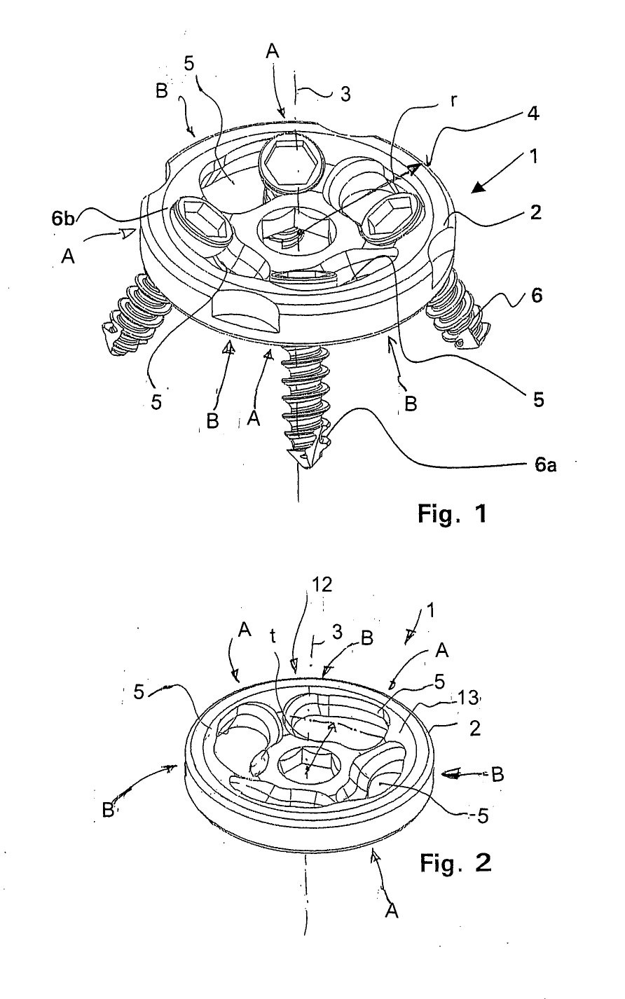

In FIGS. 1-5, an implant 1 according to one embodiment of the invention is shown, which possesses a base body 2 that is configured as a plate-shaped element. The exemplary embodiment shown here is a circular, i.e. rotation-symmetrical plate element having a central axis 3. The radius of the base body 2 is determined by the distance indicated with r, which extends from the central axis 3 all the way to the outside circumference 4 of the base body 2.

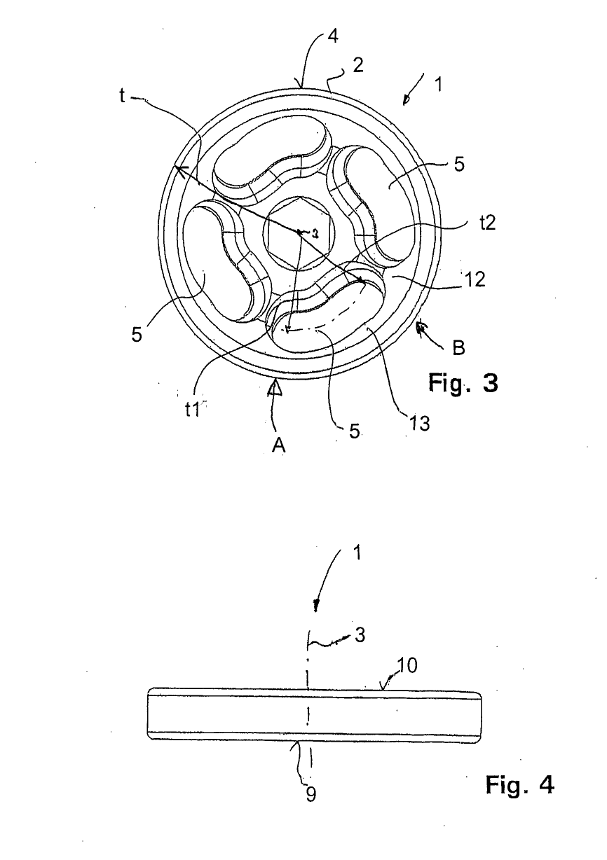

The base body 2 possesses four recesses 5 that are configured in the manner of an oblong hole. The recesses 5, in turn, have a curvature that is configured in such a manner that the radius indicated as t in FIG. 2 changes from one side A of the oblong-hole-like recess 5, in each instance, to its other side B, specifically proceeding from the point of origin on the central axis 3. Preferably, this radius t can decrease in such a manner that a kidney-like shape of the recess 5 is formed. In FIG. 3, it is shown that a radius t1 at the starting point (one side A) is greater than a radius t2 at the end point (other side B).

In this case, the curvature of each recess 5, which runs in the circumference direction of the base body 2, runs at an incline with reference to the central axis 3 of the base body 2, so that each bone screw—indicated with 6, 6a, 6b etc.—guided in the recesses 5 in the manner of a motion link can be moved radially toward the center axis 3 of the base body 2, by means of a rotational movement of the base body 2 in the direction of the inclined recesses 5 in the shape of an oblong hole, thereby exerting a tension force, in each instance, on the bone, in each instance, in the direction toward the center of the base body 2.

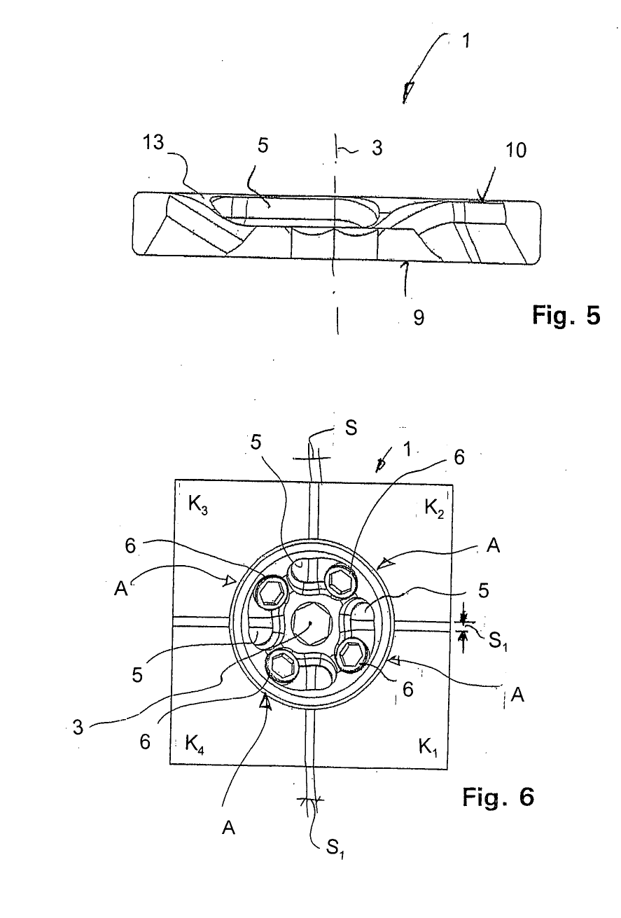

According to FIGS. 4 and 5, the implant 1 is configured in disk shape. This has the result that the side that faces the bone side—referred to as the underside 9—is configured to be level, i.e. flat, so that no conical or spherical accommodation opening, but rather simply a level depression within the bone has to be present. In order to allow flat contact, the side that faces away from the bone side—referred to as top side 10—is also configured to be flat

The recesses 5 of the implant 1 furthermore have an edge region 12 that is configured to be spherical (See FIG. 3). In this way, a surface 13 is created that corresponds to part of a mantle of a sphere. The bone screws 6, which consist of a threaded shaft 6a and a head element 6b, are to be inserted into the recesses 5. In this connection, the head element 6b should be dimensioned so that it is larger than the clear width of the recess 5, in each instance. Because the recesses 5 are delimited by a spherical surface 13, the bone screws 6 do not extend perpendicular to the implant 1 and thus parallel to the central axis 3, but rather extend perpendicularly away from the spherical surface 13. As a result, the bone screws 6 point outward and are therefore suitable for accommodating the individual bone parts.

The embodiment(s) described above can function as described in the following, using FIGS. 6 to 8:

After a level accommodation opening has been worked into the four bone parts—referred to as K1, K2, K3, and K4—the implant 1 is laid on, specifically in such a manner that the recesses 5 cover a bone part, in each instance. The bone parts K1, K2, K3, and K4 demonstrate a gap S1 from one another.

Subsequently, fixation takes place, in that the bone screws 6 are screwed into the bone parts K1 to K4 in such a manner that they are disposed in the region of the one side A of the oblong-hole-like recess 5.

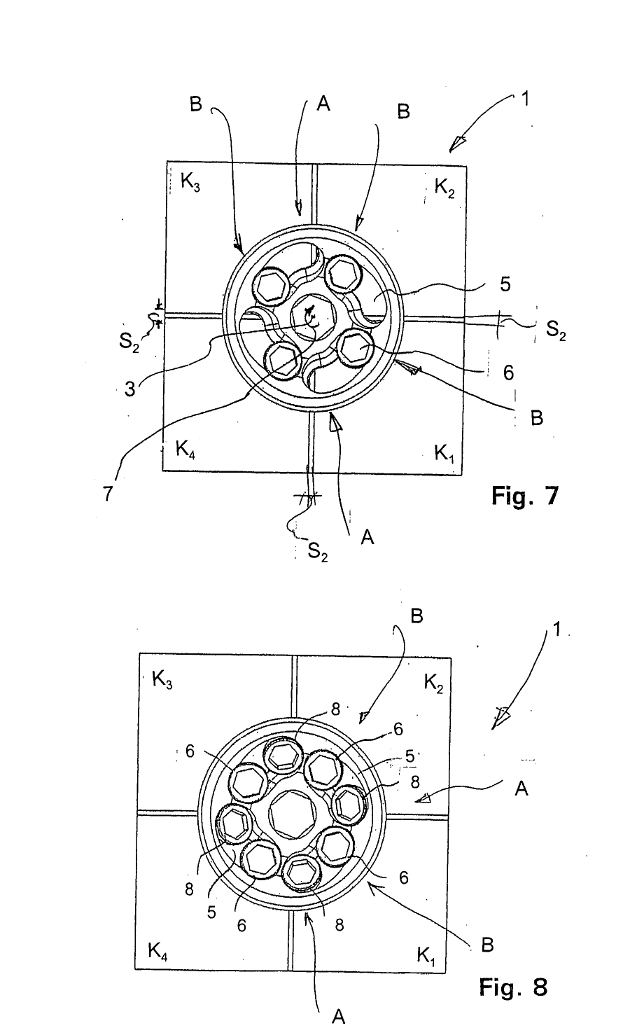

By means of subsequent rotation of the implant 1 in the arrow direction 7 (FIG. 7), the result is achieved that the bone screws 6 get from the one side A to the other end B of the recesses 5. Since the bone screws 6 fix the bone parts K1 to K4 in place, in each instance, and the recesses 5 have a curvature that points toward the center axis 3, the bone parts K1 to K4 are simultaneously guided toward the axis 3, in centered manner. As a result, in turn, the gap S1 according to FIG. 6 becomes smaller or is cancelled out, whereby the smaller gap is designated with the reference symbol S2 in FIG. 7.

To prevent the fixed position as shown in FIG. 7 from being unintentionally loosened, in the exemplary embodiment shown here, at least one other bone screw 8, in each instance, is additionally placed in the available spaces of the recesses 5 (in the region of the one end A). In this exemplary embodiment, the bone screws 8 also penetrate the recesses 5 and also penetrate the bone parts K1 to K4, at least in part. Alternatively to this, it can also be provided that in place of the bone screws 8, exclusively head screws are used that are only screwed into the implant 1, in order to prevent the bone screws 6 from moving within the recesses 5.

With the implant 1 according to one embodiment of the invention, a medical device has been created that can be used in simple manner, and can be used to achieve sufficient compression of the individual bone parts to be fused. By means of simple fixation of the corresponding bone parts K1 to K4, as well as rotation of the implants 1 about a defined angle, the individual bone parts K1 to K4 that have been incorporated are brought towards one another to such an extent that compression is achieved. Additional means, such as head screws or other bone screws, for example, serve to prevent unscrewing, i.e. decompression. Alternatively, this securing can take place by means of a suitable progression of the curvature, i.e. motion link of the recesses 5 that accommodate the screws.

The implant 1 can be produced in simple manner, since it is configured to be planar and has a simple shape of the corresponding recesses 5 for accommodating the corresponding bone screws 6 and 8, respectively. Furthermore, the number of components is limited to the actual implant 1 with the bone screws 6 and 8, respectively, as fastening means.

Accordingly, while a few embodiments of the present invention have been shown and described, it is to be understood that many changes and modifications may be made thereunto without departing from the spirit and scope of the invention as defined in the appended claims.

### REFERENCE SYMBOLS LIST

1 implant
2 base body
3 center (axis)
4 outside circumference
5 recess
6 bone screw
6a threaded shaft
6b head element
7 arrow direction
8 bone screw
9 underside
10 top side
11 - - -
12 edge region
13 spherical surface
A side
B side
K1 to K4 bone parts
r radius
t1 radius
t2 radius
S1 gap
S2 gap reduced in size

What is claimed is:

1. An implant for fusing bones or bone parts comprising:
a) a base body having a plurality of recesses;
b) a plurality of bone screws which are guided in said recesses and which have a geometry for allowing compression of the bones;
wherein said base body is configured with rotation symmetry;
and wherein said geometry of said recesses have a shape of an oblong hole curved in a circumferential direction of said base body wherein said curvature runs at an incline to said axis in such a manner that said plurality of bone screws can be moved in a direction of said inclined oblong holes radially towards said central axis.

2. The implant as in claim 1, wherein said recesses of said base body have an edge region wherein said recesses of said base body are delimited by said edge region which has a spherically configured surface that corresponds to a part of a mantle of a sphere.

3. The implant as in claim 1 wherein said base body has a planar underside.

4. The implant as in claim 1 wherein said base body has a planar topside.

5. The implant as in claim 1, wherein said base body further comprises a center recess which is configured to accommodate a shape fit turning tool.

6. The implant as in claim 1, further comprising additional screws wherein at least one recess has at least two screws positioned therein.
